(19) United States
(12) Patent Application Publication
(10) Pub. No.: US 2021/0187078 A1
(43) Pub. Date: Jun. 24, 2021

(54) PHARMACEUTICAL COMPOSITION OF INSULIN GLARGINE AND AMINO ACIDS

(71) Applicant: WOCKHARDT LIMITED, Mumbai (IN)

(72) Inventors: Maharaj Kishen SAHIB, Mumbai (IN); Ganesh BARHATE, Ahmednagar (IN); Mamatha KATRGADDA, Andhra Pradesh (IN)

(73) Assignee: WOCKHARDT LIMITED, Mumbai (IN)

(21) Appl. No.: 16/078,779

(22) PCT Filed: Feb. 24, 2017
(86) PCT No.: PCT/IB2017/051069

§ 371 Date: Aug. 22, 2018

(30) Foreign Application Priority Data
Feb. 25, 2016 (IN) 201621006633
Feb. 25, 2016 (IN) 201621006634

(51) Int. Cl. A61K 38/28; A61P 13/10

(52) U.S. Cl. CPC A61K 38/28; A61P 13/10

(57) ABSTRACT

There is provided a pharmaceutical composition comprising: (a) insulin glargine, (b) at least two amino acids, and optionally (c) one or more pharmaceutically acceptable excipients. In particular, there is provided a pharmaceutical composition comprising: (a) insulin glargine, (b) arginine and isoleucine in a weight ratio of about 1:2, and optionally (c) one or more pharmaceutically acceptable excipients.

### PRIORITY DOCUMENTS

This application claims priority from the Indian provisional application Nos. IN 201621006633 and IN 201621006634; filed on Feb 25, 2016.

### TECHNICAL FIELD OF THE INVENTION

The invention relates to pharmaceutical composition comprising insulin glargine and amino acids. In particular, the present invention relates to a pharmaceutical composition comprising: (a) insulin glargine, (b) at least two amino acids and optionally (c) one or more pharmaceutically acceptable excipients.

### BACKGROUND OF THE INVENTION

Diabetes mellitus is a metabolic disorder in which the ability to utilize glucose is more or less completely lost. For decades, insulin has been used in the treatment of diabetes mellitus for which several insulin formulations have been developed. Currently available rapid acting insulin includes insulin lispro, insulin aspart and insulin glulisine. Fast acting insulin shows faster absorption and rapid onset of action i.e. within 30 min of administration. Thus, they are used for effective control of post-prandial increase in the blood sugar level. These analogous are used for the treatment of type 1 (insulin-dependent) and type II (non-insulin-dependent) diabetes. For immediate and long term control of the glycemic level in the body, long acting insulin is given in combination with rapid acting insulin. Basal-bolus insulin therapy given as either multiple daily injections or by an insulin pump is a mainstay of diabetes treatment for achieving optimal glycemic control in type I diabetes. Attempts have been made to mix the different types of insulin in one injection in order to reduce the number of injections to be administered to a patient in the day. Due to difference in isoelectric points, mixing of different types of insulin prior to administration is not recommended as it leads to uncontrolled precipitation after mixing due to which it is difficult to predict the exact dose that has been administered by the patient.

Insulin glargine cannot be readily mixed with other insulin, insulin analogues or derivative having isoelectric points 4.0 to 5.7, because the mixture causes glargine to precipitate prior to injection and administration of precipitated insulin makes it virtually impossible to administer a known and reliable dose. Several warnings have been issued by the regulatory agencies against mixing long and rapid-acting insulin's together (insulin glargine, LANTUS; Sanofi-Aventis; available from http://www.lantus.com/hcp/closing.aspx; insulin detemir rDNA origin, brand name LEVEMIR drug insert; Novo Nordisk, Bagsvaerd, Denmark).

Kaplan W et al. (2004) discloses the effects of mixing glargine and short-acting insulin analogs on glucose control. Evans et al. (2011) discloses that neither insulin glargine nor insulin detemir are suitable for mixing with other insulin analogues as this mixing substantially alters their pharmacokinetic properties. Lucchesi M b et.al. (2012) discloses mixing Insulin Lispro with Insulin glargine immediately before the subcutaneous injection decreases Insulin Lispro serum peak concentration without affecting the glycemic profile after 12 wk in this group with type 1 diabetes mellitus. PCT publication No. WO2016/001862 A1 discloses biphasic pharmaceutical composition comprising an insulin analogue, derivative or metabolite having isoelectric point between 5.8 to 8.5, zinc or salts thereof, isotonic agent optionally along with one or more pharmaceutically acceptable excipients. PCT publication No. WO2014/118355 A1 discloses stabilized pharmaceutical formulations of insulin analogues and/or insulin derivatives. PCT publication No. WO2015/044922 A1 discloses a pharmaceutical composition comprising readily dissociable molecular aggregates formed by combining an insulin, insulin analogue, derivative or metabolite having isoelectric point between 5.8 to 8.5 in combination with one or more an insulin, insulin analogue, derivative or metabolite having isoelectric point between 4.0 to 5.7 optionally along with one or more excipients.

There is a need to develop a stable and compatible insulin glargine composition which can be readily mixed with another insulin, insulin analogue or derivative having isoelectric point between 4.0 and 5.7, and which will lead to reduction in the number of injections that a patient needs to administer.

### SUMMARY OF THE INVENTION

In one aspect of the invention, there is provided a pharmaceutical composition comprising: (a) insulin glargine, (b) at least two amino acids in a weight ratio of about 1:2, and optionally (c) one or more pharmaceutically acceptable excipients. In one embodiment, the composition has pH in between about 2 to about 8. In another embodiment, the two amino acids are selected from the group consisting of arginine, lysine, betaine, norleucine, aspartic acid, glutamic acid, asparagines, proline, glutamine, histidine, serine, threonine, alanine, leucine, isoleucine, valine, phenylalanine, tryptophan, phosphatidylethanolamine, phosphatidylserine and tyrosine. In another embodiment, the two amino acids are arginine and isoleucine. In another embodiment, the pharmaceutical composition is in the form of solution or suspension. In another embodiment, insulin glargine is present in about 40 IU to about 500 IU. In another embodiment, the one or more pharmaceutically acceptable excipients comprise buffer, solubilising agent, isotonic agent, preservative, antioxidant, pH modifying agent or combination thereof.

In another aspect of the invention, there is provided a pharmaceutical composition comprising: (a) insulin glargine, (b) at least two amino acids in a weight ratio of about 1:2, and optionally (c) one or more pharmaceutically acceptable excipients, wherein the pharmaceutical composition is compatible when mixed with another insulin, insulin analogue or derivative having isoelectric point between 4.0 and 5.7. In one embodiment, the composition has pH in between about 2 to about 8. In another embodiment, the another insulin, insulin analogue or derivative having isoelectric point between 4.0 and 5.7 is selected from the group comprising insulin human, insulin isophane (NPH), insulin lispro, insulin lispro protamine, insulin glulisine, insulin aspart, insulin aspart protamine, Gly(A21) human insulin, Gly(A21) Lys(B28) human insulin, Gly(A21) Lys(B28) Pro(B29) human insulin, Gly(A21) Asp(B28), insulin degludec and insulin detemir. In another embodiment, the two amino acids are selected from the group consisting of arginine, lysine, betaine, norleucine, aspartic acid, glutamic acid, asparagines, proline, glutamine, histidine, serine, threonine, alanine, leucine, isoleucine, valine, phenylalanine, tryptophan, phosphatidylethanolamine, phosphatidylserine and tyrosine. In another embodiment, the two amino acids are arginine and isoleucine. In another embodiment, the pharmaceutical composition is in the form of solution. In another embodiment, insulin glargine is present in about 40 IU to about 500 IU. In another embodiment, another insulin, insulin analogue or derivative having isoelectric point between 4.0 and 5.7 is present in about 40 IU to about 500 IU. In another embodiment, the one or more pharmaceutically acceptable excipients comprise buffer, solubilising agent, isotonic agent, preservative, antioxidant, pH modifying agent or combination thereof. In another embodiment, the composition is stable for at least 24 hours at 25° C. and relative humidity of 60% after mixing with another insulin, insulin analogue or derivative having isoelectric point between 4.0 and 5.7.

In another aspect of the invention, there is provided a stable biphasic pharmaceutical composition comprising: (a) insulin glargine in a suspension form, (b) another insulin, insulin analogue or derivative having isoelectric point between 4.0 and 5.7, (c) at least two amino acids in a weight ratio of about 1:2, and optionally, (d) one or more pharmaceutically acceptable excipients, wherein the pharmaceutical composition has a pH between about 6.0 to about 8.0. In another embodiment, the another insulin, insulin analogue or derivative having isoelectric point between 4.0 and 5.7 is selected from the group comprising insulin human, insulin isophane (NPH), insulin lispro, insulin lispro protamine, insulin glulisine, insulin aspart, insulin aspart protamine, Gly(A21) human insulin, Gly(A21) Lys(B28) human insulin, Gly(A21) Lys(B28) Pro(B29) human insulin, Gly(A21) Asp(B28), insulin degludec and insulin detemir. In another embodiment, the two amino acids are selected from the group consisting of arginine, lysine, betaine, norleucine, aspartic acid, glutamic acid, asparagines, proline, glutamine, histidine, serine, threonine, alanine, leucine, isoleucine, valine, phenylalanine, tryptophan, phosphatidylethanolamine, phosphatidylserine and tyrosine. In another embodiment, the two amino acids are arginine and isoleucine. In another embodiment, insulin glargine is present in about 40 IU to about 500 IU. In another embodiment, another insulin, insulin analogue or derivative having isoelectric point between 4.0 and 5.7 is present in about 40 IU to about 500 IU. In another embodiment, the one or more pharmaceutically acceptable excipients comprise buffer, solubilising agent, isotonic agent, preservative, antioxidant, pH modifying agent or combination thereof. In another embodiment, the composition is stable for at least 6 months at 25° C. and relative humidity of 60%.

In another aspect of the invention, there is provided a method of preparing a pharmaceutical composition comprising: (a) insulin glargine, (b) another insulin, insulin analogue or derivative having isoelectric point between 4.0 and 5.7, (c) at least two amino acids in a weight ratio of 1:2, and optionally (d) one or more pharmaceutically acceptable excipients, wherein the pharmaceutical composition has pH in between about 3 to about 4, and wherein the said method comprises steps of:
(i) preparing a solution comprising insulin glargine in acidic water for injection,
(ii) preparing a solution of at least two amino acids,
(iii) adding the solution of at least two amino acids in the solution of step (i) and adjusting the pH to about 3 to about 4 using a pH modifying agent,
(iv) preparing a solution comprising insulin, insulin analogous or derivative having isoelectric point between 4.0 and 5.7 having pH of about 6 to about 7, and
(v) mixing the solutions of step (iii) and (iv) to obtain the pharmaceutical composition.

In another aspect of the invention, there is provided a method of preparing a stable biphasic pharmaceutical composition comprising: (a) insulin glargine in a suspension form, (b) another insulin, insulin analogue or derivative having isoelectric point between 4.0 and 5.7, (c) at least two amino acids in a weight ratio of 1:2, and optionally (d) one or more pharmaceutically acceptable excipients, wherein the pharmaceutical composition has a pH between about 6.0 to about 8.0, wherein the said method comprises steps of:
(i) preparing a solution comprising insulin glargine having pH adjusted in between about 3 and about 4,
(ii) preparing a solution of sodium hydroxide (1N) and adding it to the solution of step (i) to obtain a suspension of insulin glargine having pH of about 7 to about 8,
(iii) preparing a solution of at least two amino acids and adding it into the suspension of step (ii) with mixing,
(iv) preparing a solution comprising insulin, insulin analogous or derivative having isoelectric point between 4.0 and 5.7 having pH of about 6 to about 7, and
(v) mixing the suspension of step (iii) and solution of step (iv) to obtain the stable biphasic pharmaceutical composition.

In another aspect of the invention, there is provided a method of treating type I and type II diabetes mellitus in a patient in need thereof, said method comprises administering to said patient a pharmaceutical composition comprising: (a) insulin glargine, (b) at least two amino acids in a weight ratio of about 1:2, and optionally (c) one or more pharmaceutically acceptable excipients.

In another aspect of the invention, there is provided a method of treating type I and type II diabetes mellitus in a patient in need thereof, said method comprises administering to said patient a pharmaceutical composition comprising: (a) insulin glargine, (b) at least two amino acids in a weight ratio of about 1:2, and optionally (c) one or more pharmaceutically acceptable excipients, wherein the pharmaceutical composition is compatible when mixed with another insulin, insulin analogue or derivative having isoelectric point between 4.0 and 5.7.

In another aspect of the invention, there is provided a method of treating type I and type II diabetes mellitus in a patient in need thereof, said method comprises administering to said patient a stable biphasic pharmaceutical composition comprising: (a) insulin glargine in a suspension form, (b) another insulin, insulin analogue or derivative having isoelectric point between 4.0 and 5.7, (c) at least two amino acids in a weight ratio of about 1:2, and optionally (d) one or more pharmaceutically acceptable excipients, wherein the pharmaceutical composition has a pH between about 6.0 to about 8.0.

In another aspect of the invention, there is provided a kit comprising first and second component, said first component comprises a pharmaceutical composition comprising: (a) insulin glargine, (b) at least two amino acids in a weight ratio of about 1:2, and optionally (c) one or more pharmaceutically acceptable excipients, and said second component comprises a pharmaceutical composition comprising: (a) another insulin, insulin analogue or derivative having isoelectric point between 4.0 and 5.7 and (b) one or more pharmaceutically acceptable excipients, and wherein the pharmaceutical composition of the first component is compatible when mixed with the pharmaceutical composition of second component.

### DETAILED DESCRIPTION OF THE INVENTION

In general aspect of the invention, there is provided a pharmaceutical composition comprising: (a) insulin glargine, (b) at least two amino acids, and optionally (c) one or more pharmaceutically acceptable excipients.

The term “insulin” herein includes mammalian insulin, insulin human.

The term “insulin analogue” herein includes insulin NPH, insulin lispro, insulin lispro protamine, insulin glulisine, insulin aspart, insulin aspart protamine, Gly(A21) human insulin, Gly(A21) Lys(B28) human insulin, Gly(A21) Lys(B28) Pro(B29) human insulin, Gly(A21) Asp(B28).

The term “insulin derivative” herein includes B29-Nεmyristoyl-des(B30) human insulin (insulin detemir), Lys(B29)-Nε-(N-palmitoyl-.gamma.-glutamyl)-des(B30) human insulin (insulin degludec), B29-Nε-palmitoyl-des(B30) human insulin, B29-Nε-myristoyl human insulin, B29-Nε-palmitoyl human insulin, B28-Nε-myristoyl LysB28ProB29 human insulin, B28-Nε-palmitoyl LysB28ProB29 human insulin, B30-Nε-myristoyl-ThrB29LysB30 human insulin, B30-Nε-palmitoylThrB29LysB30 human insulin, B29-Nε-(N-lithocholyl-.gamma.-glutamyl)-des(B30) human insulin, B29-Nε-b(ω-carboxyheptadecanoyl)des(B30) human insulin, B29-Nε-(ω-carboxyheptadecanoyl) human insulin.

The term “stable” herein relates to a physical and/or chemical stability of pharmaceutical composition of insulin.

The term “compatible” herein relates to a physical and/or chemical compatibility of insulin glargine composition when mixed with another insulin, insulin analogue or derivative having isoelectric point between 4.0 and 5.7.

The term “biphasic” herein relates to a composition of insulin glargine and another insulin, insulin analogue or derivative having isoelectric point between 4.0 and 5.7, wherein insulin glargine is in suspension form and another insulin, insulin analogue or derivative having isoelectric point between 4.0 and 5.7 is in solution form.

The term “pharmaceutically acceptable excipients” herein relates to non-active pharmaceutical ingredients which are within the scope of sound medical judgment suitable for use in pharmaceutical products.

The term “isoelectric point” herein relates is the pH at which the insulin molecule carries no net electrical charge in the statistical mean.

For clarity, insulin glargine of the present invention can also be denoted as Gly(A21), Arg(B31), Arg(B32) insulin human and insulin aspart of the present invention can also be denoted as Asp(B28) insulin human.

In one aspect of the invention, there is provided a pharmaceutical composition comprising: (a) insulin glargine, (b) at least two amino acids in a weight ratio of about 1:2, and optionally (c) one or more pharmaceutically acceptable excipients.

In another aspect of the invention, there is provided a pharmaceutical composition comprising: (a) insulin glargine, (b) at least two amino acids in a weight ratio of about 1:2, and optionally (c) one or more pharmaceutically acceptable excipients, wherein the pharmaceutical composition is compatible when mixed with another insulin, insulin analogue or derivative having isoelectric point between 4.0 and 5.7.

In another aspect of the invention, there is provided a stable biphasic pharmaceutical composition comprising: (a) insulin glargine in a suspension form, (b) another insulin, insulin analogue or derivative having isoelectric point between 4.0 and 5.7, (c) at least two amino acids in a weight ratio of about 1:2, and optionally, (d) one or more pharmaceutically acceptable excipients, wherein the pharmaceutical composition has a pH between about 6.0 to about 8.0.

In one embodiment, the composition has a pH in between about 2 to about 8. Alternatively, the composition has pH in between about 3 to about 7, or about 3 to about 4, or about 6 to about 8. Each of the pH range constitutes an alternate embodiment of the invention.

In another embodiment, the two amino acids are selected from the group consisting of arginine, lysine, betaine, norleucine, aspartic acid, glutamic acid, asparagines, proline, glutamine, histidine, serine, threonine, alanine, leucine, isoleucine, valine, phenylalanine, tryptophan, phosphatidylethanolamine, phosphatidylserine and tyrosine. Each amino acid constitutes an alternate embodiment of the invention. In another embodiment, the two amino acids are arginine and isoleucine. In an embodiment, the concentration of arginine or isoleucine ranges from about 1 mM to 100 mM, or about 2 mM to 50 mM, or about 3 mM to 25 mM. In an embodiment, the concentration of arginine is about 2 mM to 20 mM, or about 2 mM to 10 mM. Alternatively, the concentration of arginine is about 3 mM, or about 4 mM, or about 5 mM, or about 6 mM, or about 7 mM, or about 8 mM, or about 9 mM. In an embodiment, the concentration of isoleucine is about 2 mM to 20 mM, or about 2 mM to 15 mM. Alternatively, the concentration of isoleucine is about 3 mM, or about 4 mM, or about 5 mM, or about 6 mM, or about 7 mM, or about 8 mM, or about 9 mM, or about 10 mM, or about 11 mM, or about 12 mM, or about 13 mM, or about 14 mM. Alternatively, the concentration of arginine is about 7 mM and the concentration of isoleucine is about 14 mM.

In another embodiment, the pharmaceutical composition is in the form of solution or suspension. In still another embodiment, the pharmaceutical composition is suitable for parenteral administration such as intramuscular, subcutaneous, intradermal and intravenous administration.

In another embodiment, the another insulin, insulin analogue or derivative having isoelectric point between 4.0 and 5.7 is selected from the group comprising insulin human, insulin isophane (NPH), insulin lispro, insulin lispro protamine, insulin glulisine, insulin aspart, insulin aspart protamine, Gly(A21) human insulin, Gly(A21) Lys(B28) human insulin, Gly(A21) Lys(B28) Pro(B29) human insulin, Gly(A21) Asp(B28), insulin degludec and insulin detemir. Alternatively, the another insulin, insulin analogue or derivative having isoelectric point between 4.0 and 5.7 is insulin human or insulin aspart or insulin lispro.

In another embodiment, insulin glargine is present in about 40 IU to about 500 IU. Alternatively, insulin glargine is present in about 80 IU to about 400 IU, or about 100 IU to about 300 IU, or about 200 IU to about 300 IU. Alternatively, insulin glargine is present in about 50 IU, or about 60 IU, or about 70 IU, or about 80 IU, or about 90 IU, or about 100 IU, or about 110 IU, or about 120 IU, or about 130 IU, or about 140 IU, or about 150 IU, or about 160 IU, or about 170 IU, or about 180 IU, or about 190 IU, or about 200 IU, or about 250 IU, or about 300 IU, or about 350 IU, or about 400 IU, or about 450 IU. Each of this concentration constitutes an alternate embodiment of the invention. In another embodiment, another insulin, insulin analogue or derivative having isoelectric point between 4.0 and 5.7 is present in about 40 IU to about 500 IU. Alternatively, another insulin, insulin analogue or derivative having isoelectric point between 4.0 and 5.7 (e.g., insulin human or insulin aspart or insulin lispro) is present in about 80 IU to about 400 IU, or about 100 IU to about 300 IU, or about 200 IU to about 300 IU. Alternatively, another insulin, insulin analogue or derivative having isoelectric point between 4.0 and 5.7 is present in about 50 IU, or about 60 IU, or about 70 IU, or about 80 IU, or about 90 IU, or about 100 IU, or about 110 IU, or about 120 IU, or about 130 IU, or about 140 IU, or about 150 IU, or about 160 IU, or about 170 IU, or about 180 IU, or about 190 IU, or about 200 IU, or about 250 IU, or about 300 IU, or about 350 IU, or about 400 IU, or about 450 IU. Each of this concentration constitutes an alternate embodiment of the invention.

In an embodiment, the compositions of insulin glargine and another insulin, insulin analogue or derivative having isoelectric point between 4.0 and 5.7 (e.g., insulin human or insulin aspart or insulin lispro) are ready to be mixed with each other before injecting into a patient in the proportion ranging from, as percentage, 90:10 to 50:50. Alternatively, the compositions are ready to be mixed with each other in the proportion, as percentage, 85:15, or 80:20, or 75:25, or 70:30, 65:35, or 60:40, or 55:45. In another embodiment, such composition (mixed before injection) is stable for at least 24 hours at 25° C. and relative humidity of 60% after mixing with another insulin, insulin analogue or derivative having isoelectric point between 4.0 and 5.7.

Alternatively, the compositions of insulin glargine and another insulin, insulin analogue or derivative having isoelectric point between 4.0 and 5.7 (eg., insulin human or insulin aspart or insulin lispro) can be provided in the form of a stable and biphasic composition and in fixed dose proportion, as percentage, 90:10, or 85:15, or 80:20, or 75:25, or 70:30, 65:35, or 60:40, or 55: 45 or 50:50. In another embodiment, such fixed dose composition is stable for at least 6 months at 25° C. and relative humidity of 60%.

In another embodiment, the stable and biphasic composition comprises insulin glargine in suspension form and wherein insulin glargine possess particle size (D90) ranging from 2 μm to 60 μm, or about 5 μm to 30 μm, or about 10 μm to 20 μm.

In another embodiment, the one or more pharmaceutically acceptable excipients comprise buffer, solubilising agent, isotonic agent, preservative, antioxidant, pH modifying agent or combination thereof.

The pH modifying agents as used herein refers to a combination of acid and alkali. The pH modifying agents can be selected from the group comprising of hydrochloric acid, o-phosphoric acid, citric acid, acetic acid, succinic acid, lactic acid, gluconic acid, tartaric acid, 1,2,3,4-butane tetracarboxylic acid, fumaric acid or malic acid Alkali is selected from the group comprising of sodium hydroxide, potassium hydroxide, sodium hydroxide, ammonium hydroxide, magnesium oxide, calcium hydroxide, calcium carbonate, magnesium carbonate, magnesium aluminum silicates, diethanolamine, monoethanolamine, sodium carbonate, sodium bicarbonate or triethanolamine and combination thereof.

The solubilizing agent are selected from the group consisting of partial and fatty acid esters and ethers of polyhydric alcohols such as of glycerol, sorbitol, glycine and the like (Span®, Tween®, in particular Tween® 20 and Tween®80, Myrj®, Brij®, Cremophore® or poloxamers, Pluronics® and Tetronics®), polysorbates (Tween™), sodium dodecyl sulfate (sodium lauryl sulfate), lauryl dimethyl amine oxide, cetyltrimethylammonium bromide (CTAB), polyethoxylated alcohols polyoxyethylene sorbitan, Octoxynol (Triton X100™), N, N-dimethyldodecylamine-N-oxide, hexadecyltrimethylammonium bromide (HTAB), polyoxyl 10 lauryl ether, Brij 721™, bile salts (sodium deoxycholate, sodium cholate), polyoxyl castor oil (Cremophor™), nonylphenol ethoxylate (Tergitol™), cyclodextrins, lecithin and methylbenzethonium chloride (Hyamine™). The concentration of solubilizing agent (preferred is glycine) is about 1 mM to 200 mM or about 70 mM, or about 100 mM.

The isotonic agent as used herein includes salts, e.g., sodium chloride, dextrose, lactose or combination thereof.

The preservative as used herein include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, 2-penoxyethanol, phenyl mercuric nitrate, thimerosal, metacresol and combinations thereof. The concentration of preservative is about 0.1 mg/mL to about 5 mg/mL, or about 0.4 mg/ml, or about 2.4 mg/ml.

The antioxidant as used herein can be selected from the group comprising of ascorbate (sodium/acid), bisulite sodium, butylated hydroxy anisole (BHA), butylated hydroxy toluene (BHT), cysteine/cysteinate HCl, dithionite sodium, gentisic acid, gentisic acid ethanolamine, glutamate monosodium, glutathione, formaldehyde sulfoxylate sodium, metabisulite potassium, metabisulite sodium, methionine, monothioglycerol (thioglycerol), propyl gallate, sulfite sodium, tocopherol alpha, alpha tocopherol hydrogen succinate, thioglycolate sodium and combination thereof.

The buffer as used herein include, but are not limited to, phosphate, acetate, citrate or TRIS (i.e. 2-amino-2-hydroxymethyl-1,3-propanediol) buffer and corresponding salts.

In one embodiment of the invention, there is provided a pharmaceutical composition comprising: (a) insulin glargine, (b) arginine and isoleucine in a weight ratio of about 1:2, and optionally (c) one or more pharmaceutically acceptable excipients.

In one embodiment of the invention, there is provided a pharmaceutical composition comprising: (a) insulin glargine, (b) arginine and isoleucine in a weight ratio of about 1:2, and optionally (c) one or more pharmaceutically acceptable excipients, and wherein the composition has pH in between about 2 to about 8, and wherein the pharmaceutical composition is in the form of solution or suspension, and wherein insulin glargine is present in about 40 IU to about 500 IU, and wherein the one or more pharmaceutically acceptable excipients comprise buffer, solubilising agent, isotonic agent, preservative, antioxidant, pH modifying agent or combination thereof.

In one embodiment of the invention, there is provided a pharmaceutical composition comprising: (a) insulin glargine, (b) arginine and isoleucine in a weight ratio of about 1:2, and optionally (c) one or more pharmaceutically acceptable excipients, and wherein the composition has pH in between about 2 to about 8, and wherein the pharmaceutical composition is in the form of solution or suspension, and wherein insulin glargine is present in about 50 IU, or about 60 IU, or about 70 IU, or about 80 IU, or about 90 IU, or about 100 IU, or about 110 IU, or about 120 IU, or about 130 IU, or about 140 IU, or about 150 IU, or about 160 IU, or about 170 IU, or about 180 IU, or about 190 IU, or about 200 IU, or about 250 IU, or about 3001 U, or about 350 IU, or about 400 IU, or about 450 IU, and wherein the one or more pharmaceutically acceptable excipients comprise buffer, solubilising agent, isotonic agent, preservative, antioxidant, pH modifying agent or combination thereof.

In another embodiment of the invention, there is provided a pharmaceutical composition comprising: (a) insulin glargine, (b) two amino acids in a weight ratio of about 1:2, and optionally (c) one or more pharmaceutically acceptable excipients, wherein the pharmaceutical composition is compatible when mixed with another insulin, insulin analogue or derivative having isoelectric point between 4.0 and 5.7.

In another embodiment of the invention, there is provided a pharmaceutical composition comprising: (a) insulin glargine, (b) arginine and isoleucine in a weight ratio of about 1:2, and optionally (c) one or more pharmaceutically acceptable excipients, wherein the pharmaceutical composition is compatible when mixed with another insulin, insulin analogue or derivative having isoelectric point between 4.0 and 5.7.

In another embodiment of the invention, there is provided a pharmaceutical composition comprising: (a) insulin glargine, (b) arginine and isoleucine in a weight ratio of about 1:2, and optionally (c) one or more pharmaceutically acceptable excipients, wherein the pharmaceutical composition is compatible when insulin human or insulin aspart.

In another embodiment of the invention, there is provided a pharmaceutical composition comprising: (a) insulin glargine, (b) arginine and isoleucine in a weight ratio of about 1:2, and optionally (c) one or more pharmaceutically acceptable excipients, wherein the pharmaceutical composition is compatible when mixed with another insulin, insulin analogue or derivative having isoelectric point between 4.0 and 5.7, and wherein the composition has pH in between about 2 to about 8, and wherein the another insulin, insulin analogue or derivative having isoelectric point between 4.0 and 5.7 is selected from the group comprising insulin human, insulin isophane (NPH), insulin lispro, insulin lispro protamine, insulin glulisine, insulin aspart, insulin aspart protamine, Gly(A21) human insulin, Gly(A21) Lys(B28) human insulin, Gly(A21) Lys(B28) Pro(B29) human insulin, Gly(A21) Asp(B28), insulin degludec and insulin detemir, and wherein the pharmaceutical composition is in the form of solution, and wherein insulin glargine is present in about 40 IU to about 500 IU and another insulin, insulin analogue or derivative having isoelectric point between 4.0 and 5.7 is present in about 40 IU to about 500 IU, and wherein the one or more pharmaceutically acceptable excipients comprise buffer, solubilising agent, isotonic agent, preservative, antioxidant, pH modifying agent or combination thereof, and wherein the composition is stable for at least 24 hours at 25° C. and relative humidity of 60% after mixing with another insulin, insulin analogue or derivative having isoelectric point between 4.0 and 5.7.

In another embodiment of the invention, there is provided a pharmaceutical composition comprising: (a) insulin glargine, (b) arginine and isoleucine in a weight ratio of about 1:2, and optionally (c) one or more pharmaceutically acceptable excipients, wherein the pharmaceutical composition is compatible when mixed with another insulin, insulin analogue or derivative having isoelectric point between 4.0 and 5.7, and wherein the composition has pH in between about 2 to about 8, and wherein the another insulin, insulin analogue or derivative having isoelectric point between 4.0 and 5.7 is selected from the group comprising insulin human, insulin isophane (NPH), insulin lispro, insulin lispro protamine, insulin glulisine, insulin aspart, insulin aspart protamine, Gly(A21) human insulin, Gly(A21) Lys(B28) human insulin, Gly(A21) Lys(B28) Pro(B29) human insulin, Gly(A21) Asp(B28), insulin degludec and insulin detemir, and wherein the pharmaceutical composition is in the form of solution, and wherein insulin glargine is present in about 50 IU, or about 60 IU, or about 70 IU, or about 80 IU, or about 90 IU, or about 100 IU, or about 110 IU, or about 120IU, or about 130 IU, or about 140 IU, or about 150 IU, or about 160 IU, or about 170 IU, or about 180IU, or about 190 IU, or about 200 IU, or about 250 IU, or about 300 IU, or about 350 IU, or about 400 IU, or about 450 IU, and another insulin, insulin analogue or derivative having isoelectric point between 4.0 and 5.7 is present in about 50 IU, or about 60 IU, or about 70 IU, or about 80 IU, or about 90 IU, or about 100 IU, or about 110 IU, or about 120 IU, or about 130 IU, or about 140 IU, or about 150 IU, or about 160 IU, or about 170 IU, or about 180 IU, or about 190 IU, or about 200 IU, or about 250 IU, or about 300 IU, or about 350 IU, or about 400 IU, or about 450 IU, and wherein the one or more pharmaceutically acceptable excipients comprise buffer, solubilising agent, isotonic agent, preservative, antioxidant, pH modifying agent or combination thereof, and wherein the composition is stable for at least 24 hours at 25° C. and relative humidity of 60% after mixing with another insulin, insulin analogue or derivative having isoelectric point between 4.0 and 5.7.

In another embodiment of the invention, there is provided a pharmaceutical composition comprising: (a) insulin glargine, (b) arginine and isoleucine in a weight ratio of about 1:2, and optionally (c) one or more pharmaceutically acceptable excipients, wherein the pharmaceutical composition is compatible when mixed with another insulin, insulin analogue or derivative having isoelectric point between 4.0 and 5.7, and wherein the composition of insulin glargine can be mixed with another insulin, insulin analogue or derivative having isoelectric point between 4.0 and 5.7 before injecting into a patient in the proportion ranging from, as percentage, 90:10 to 50:50.

In another embodiment of the invention, there is provided a pharmaceutical composition comprising: (a) insulin glargine, (b) arginine and isoleucine in a weight ratio of about 1:2, and optionally (c) one or more pharmaceutically acceptable excipients, wherein the pharmaceutical composition is compatible when mixed with another insulin, insulin analogue or derivative having isoelectric point between 4.0 and 5.7, and wherein the composition of insulin glargine can be mixed with another insulin, insulin analogue or derivative having isoelectric point between 4.0 and 5.7 before injecting into a patient in the proportion, as percentage, 85:15, or 80:20, or 75:25, or 70:30, 65:35, or 60:40, or 55:45.

In another embodiment of the invention, there is provided a pharmaceutical composition comprising: (a) insulin glargine, (b) arginine and isoleucine in a weight ratio of about 1:2, and optionally (c) one or more pharmaceutically acceptable excipients, wherein the pharmaceutical composition is compatible when mixed with insulin human or insulin aspart, and wherein the composition of insulin glargine can be mixed with insulin human or insulin aspart before injecting into a patient in the proportion, as percentage, 85:15, or 80:20, or 75:25, or 70:30, 65:35, or 60:40, or 55:45.

In another embodiment of the invention, there is provided a pharmaceutical composition comprising: (a) insulin glargine, (b) arginine and isoleucine in a weight ratio of about 1:2, and optionally (c) one or more pharmaceutically acceptable excipients, wherein the pharmaceutical composition is compatible when mixed with insulin human or insulin aspart, and wherein the composition of insulin glargine can be mixed with insulin human or insulin aspart before injecting into a patient in the proportion, as percentage, 85:15, or 80:20, or 75:25, or 70:30, 65:35, or 60:40, or 55:45, and wherein the composition has a pH in between about 3 to about 4.

In another embodiment of the invention, there is provided a pharmaceutical composition comprising: (a) insulin glargine, (b) arginine and isoleucine in a weight ratio of about 1:2, and optionally (c) one or more pharmaceutically acceptable excipients, wherein the pharmaceutical composition is compatible when mixed with insulin human or insulin aspart, and wherein the composition of insulin glargine can be mixed with insulin human or insulin aspart before injecting into a patient in the proportion, as percentage, 85:15, or 80:20, or 75:25, or 70:30, 65:35, or 60:40, or 55:45, and wherein the composition has a pH in between about 3 to about 4, and wherein the composition is stable for at least 24 hours at 25° C. and relative humidity of 60% after mixing with insulin human or insulin aspart.

In another embodiment of the invention, there is provided a stable biphasic pharmaceutical composition comprising: (a) insulin glargine in a suspension form, (b) another insulin, insulin analogue or derivative having isoelectric point between 4.0 and 5.7, (c) two amino acids in a weight ratio of about 1:2, and optionally, (d) one or more pharmaceutically acceptable excipients, wherein the pharmaceutical composition has a pH between about 6.0 to about 8.0.

In another embodiment of the invention, there is provided a stable biphasic pharmaceutical composition comprising: (a) insulin glargine in a suspension form, (b) another insulin, insulin analogue or derivative having isoelectric point between 4.0 and 5.7, (c) arginine and isoleucine in a weight ratio of about 1:2, and optionally (d) one or more pharmaceutically acceptable excipients, wherein the pharmaceutical composition has a pH between about 6.0 to about 8.0.

In another embodiment of the invention, there is provided a stable biphasic pharmaceutical composition comprising: (a) insulin glargine in a suspension form, (b) insulin aspart, (c) arginine and isoleucine in a weight ratio of about 1:2, and optionally (d) one or more pharmaceutically acceptable excipients, wherein the pharmaceutical composition has a pH between about 6.0 to about 8.0.

In another embodiment of the invention, there is provided a stable biphasic pharmaceutical composition comprising: (a) insulin glargine in a suspension form, (b) insulin human, (c) arginine and isoleucine in a weight ratio of about 1:2, and optionally (d) one or more pharmaceutically acceptable excipients, wherein the pharmaceutical composition has a pH between about 6.0 to about 8.0.

In another embodiment of the invention, there is provided a stable biphasic pharmaceutical composition comprising: (a) insulin glargine in a suspension form, (b) another insulin, insulin analogue or derivative having isoelectric point between 4.0 and 5.7, (c) arginine and isoleucine in a weight ratio of about 1:2, and optionally (d) one or more pharmaceutically acceptable excipients, wherein the pharmaceutical composition has a pH between about 6.0 to about 8.0, and wherein the another insulin, insulin analogue or derivative having isoelectric point between 4.0 and 5.7 is selected from the group comprising insulin human, insulin isophane (NPH), insulin lispro, insulin lispro protamine, insulin glulisine, insulin aspart, insulin aspart protamine, Gly(A21) human insulin, Gly(A21) Lys(B28) human insulin, Gly(A21) Lys(B28) Pro(B29) human insulin, Gly(A21) Asp(B28), insulin degludec and insulin detemir, and wherein insulin glargine is present in about 40 IU to about 500 IU, and wherein another insulin, insulin analogue or derivative having isoelectric point between 4.0 and 5.7 is present in about 40 IU to about 500 IU, and wherein the one or more pharmaceutically acceptable excipients comprise buffer, solubilising agent, isotonic agent, preservative, antioxidant, pH modifying agent or combination thereof, and wherein the composition is stable for at least 6 months at 25° C. and relative humidity of 60%.

In another embodiment of the invention, there is provided a stable biphasic pharmaceutical composition comprising: (a) insulin glargine in a suspension form, (b) another insulin, insulin analogue or derivative having isoelectric point between 4.0 and 5.7, (c) arginine and isoleucine in a weight ratio of about 1:2, and optionally (d) one or more pharmaceutically acceptable excipients, wherein the pharmaceutical composition has a pH between about 6.0 to about 8.0, and wherein the another insulin, insulin analogue or derivative having isoelectric point between 4.0 and 5.7 is selected from the group comprising insulin human, insulin isophane (NPH), insulin lispro, insulin lispro protamine, insulin glulisine, insulin aspart, insulin aspart protamine, Gly(A21) human insulin, Gly(A21) Lys(B28) human insulin, Gly(A21) Lys(B28) Pro(B29) human insulin, Gly(A21) Asp(B28), insulin degludec and insulin detemir, and wherein insulin glargine is present in about 50 IU, or about 60 IU, or about 70 IU, or about 80 IU, or about 90 IU, or about 100 IU, or about 110 IU, or about 120 IU, or about 130 IU, or about 140 IU, or about 150 IU, or about 160 IU, or about 170 IU, or about 180 IU, or about 190 IU, or about 200 IU, or about 250 IU, or about 300 IU, or about 350 IU, or about 400 IU, or about 450 IU, and wherein another insulin, insulin analogue or derivative having isoelectric point between 4.0 and 5.7 is present in about 50 IU, or about 60 IU, or about 70 IU, or about 80 IU, or about 90 IU, or about 100 IU, or about 110 IU, or about 120 IU, or about 130 IU, or about 140 IU, or about 150 IU, or about 160 IU, or about 170 IU, or about 180 IU, or about 190 IU, or about 200 IU, or about 250 IU, or about 300 IU, or about 350 IU, or about 400 IU, or about 45 IU, and wherein the one or more pharmaceutically acceptable excipients comprise buffer, solubilising agent, isotonic agent, preservative, antioxidant, pH modifying agent or combination thereof, and wherein the composition is stable for at least 6 months at 25° C. and relative humidity of 60%.

In another embodiment of the invention, there is provided a stable biphasic pharmaceutical composition comprising: (a) insulin glargine in a suspension form, (b) another insulin, insulin analogue or derivative having isoelectric point between 4.0 and 5.7, (c) arginine and isoleucine in a weight ratio of about 1:2, and optionally (d) one or more pharmaceutically acceptable excipients, wherein the pharmaceutical composition has a pH between about 6.0 to about 8.0, and wherein insulin glargine and another insulin, insulin analogue or derivative having isoelectric point between 4.0 and 5.7 are present in a fixed dose proportion, as percentage, 90:10, or 85:15, or 80:20, or 75:25, or 70:30, 65:35, or 60:40, or 55: 45 or 50:50.

In another embodiment of the invention, there is provided a stable biphasic pharmaceutical composition comprising: (a) insulin glargine in a suspension form, (b) insulin aspart, (c) arginine and isoleucine in a weight ratio of about 1:2, and optionally (d) one or more pharmaceutically acceptable excipients, wherein the pharmaceutical composition has a pH between about 6.0 to about 8.0, and wherein insulin glargine and insulin aspart are present in a fixed dose proportion, as percentage, 90:10, or 85:15, or 80:20, or 75:25, or 70:30, 65:35, or 60:40, or 55:45 or 50:50.

In another embodiment of the invention, there is provided a stable biphasic pharmaceutical composition comprising: (a) insulin glargine in a suspension form, (b) insulin human, (c) arginine and isoleucine in a weight ratio of about 1:2, and optionally (d) one or more pharmaceutically acceptable excipients, wherein the pharmaceutical composition has a pH between about 6.0 to about 8.0, and wherein insulin glargine and insulin human are present in a fixed dose proportion, as percentage, 90:10, or 85:15, or 80:20, or 75:25, or 70:30, 65:35, or 60:40, or 55:45 or 50:50.

In another embodiment of the invention, there is provided a stable biphasic pharmaceutical composition comprising: (a) insulin glargine in a suspension form, (b) insulin aspart, (c) arginine and isoleucine in a weight ratio of about 1:2, and optionally (d) one or more pharmaceutically acceptable excipients, wherein the pharmaceutical composition has a pH between about 6.0 to about 8.0, and wherein insulin glargine and insulin aspart are present in a fixed dose proportion, as percentage, 90:10, or 85:15, or 80:20, or 75:25, or 70:30, 65:35, or 60:40, or 55: 45 or 50:50, and wherein the composition is stable for at least 6 months at 25° C. and relative humidity of 60%.

In another embodiment of the invention, there is provided a stable biphasic pharmaceutical composition comprising: (a) insulin glargine in a suspension form, (b) insulin human, (c) arginine and isoleucine in a weight ratio of about 1:2, and optionally (d) one or more pharmaceutically acceptable excipients, wherein the pharmaceutical composition has a pH between about 6.0 to about 8.0, and wherein insulin glargine and insulin human are present in a fixed dose proportion, as percentage, 90:10, or 85:15, or 80:20, or 75:25, or 70:30, 65:35, or 60:40, or 55: 45 or 50:50, and wherein the composition is stable for at least 6 months at 25° C. and relative humidity of 60%.

In another embodiment of the invention, there is provided a method of preparing a pharmaceutical composition comprising: (a) insulin glargine, (b) another insulin, insulin analogue or derivative having isoelectric point between 4.0 and 5.7, (c) at least two amino acids in a weight ratio of 1:2, and optionally (d) one or more pharmaceutically acceptable excipients, wherein the pharmaceutical composition has pH in between about 3 to about 4, and wherein the said method comprises steps of:
(i) preparing a solution comprising insulin glargine in acidic water for injection,
(ii) preparing a solution of at least two amino acids,
(iii) adding the solution of at least two amino acids in the solution of step (i) and adjusting the pH to about 3 to about 4 using a pH modifying agent,
(iv) preparing a solution comprising insulin, insulin analogous or derivative having isoelectric point between 4.0 and 5.7 having pH of about 6 to about 7, and
(v) mixing the solutions of step (iii) and (iv) to obtain the pharmaceutical composition.

In another embodiment of the invention, there is provided a method of preparing a pharmaceutical composition comprising: (a) insulin glargine, (b) insulin aspart, (c) at least two amino acids in a weight ratio of 1:2, and optionally (d) one or more pharmaceutically acceptable excipients, wherein the pharmaceutical composition has pH in between about 3 to about 4, and wherein the said method comprises steps of:
(i) preparing a solution comprising insulin glargine in acidic water for injection,
(ii) preparing a solution of at least two amino acids,
(iii) adding the solution of at least two amino acids in the solution of step (i) and adjusting the pH to about 3 to about 4 using a pH modifying agent,
(iv) preparing a solution comprising insulin aspart having pH of about 6 to about 7, and
(v) mixing the solutions of step (iii) and (iv) to obtain the pharmaceutical composition.

In another embodiment of the invention, there is provided a method of preparing a pharmaceutical composition comprising: (a) insulin glargine, (b) insulin human, (c) at least two amino acids in a weight ratio of 1:2, and optionally (d) one or more pharmaceutically acceptable excipients, wherein the pharmaceutical composition has pH in between about 3 to about 4, and wherein the said method comprises steps of:
(i) preparing a solution comprising insulin glargine in acidic water for injection,
(ii) preparing a solution of at least two amino acids,
(iii) adding the solution of at least two amino acids in the solution of step (i) and adjusting the pH to about 3 to about 4 using a pH modifying agent,
(iv) preparing a solution comprising insulin human having pH of about 6 to about 7, and
(v) mixing the solutions of step (iii) and (iv) to obtain the pharmaceutical composition.

In another embodiment of the invention, there is provided a method of preparing a stable biphasic pharmaceutical composition comprising: (a) insulin glargine in a suspension form, (b) another insulin, insulin analogue or derivative having isoelectric point between 4.0 and 5.7, (c) at least two amino acids in a weight ratio of 1:2, and optionally (d) one or more pharmaceutically acceptable excipients, wherein the pharmaceutical composition has a pH between about 6.0 to about 8.0, wherein the said method comprises steps of:
(i) preparing a solution comprising insulin glargine having pH adjusted in between about 3 and about 4,
(ii) preparing a solution of sodium hydroxide (1N) and adding it to the solution of step (i) to obtain a suspension of insulin glargine having pH of about 7 to about 8,
(iii) preparing a solution of at least two amino acids and adding it into the suspension of step (ii) with mixing,
(iv) preparing a solution comprising insulin, insulin analogous or derivative having isoelectric point between 4.0 and 5.7 having pH of about 6 to about 7, and
(v) mixing the suspension of step (iii) and solution of step (iv) to obtain the stable biphasic pharmaceutical composition.

In another embodiment of the invention, there is provided a method of preparing a stable biphasic pharmaceutical composition comprising: (a) insulin glargine in a suspension form, (b) insulin aspart, (c) at least two amino acids in a weight ratio of 1:2, and optionally (d) one or more pharmaceutically acceptable excipients, wherein the pharmaceutical composition has a pH between about 6.0 to about 8.0, wherein the said method comprises steps of:
(i) preparing a solution comprising insulin glargine having pH adjusted in between about 3 and about 4,
(ii) preparing a solution of sodium hydroxide (1N) and adding it to the solution of step (i) to obtain a suspension of insulin glargine having pH of about 7 to about 8,
(iii) preparing a solution of at least two amino acids and adding it into the suspension of step (ii) with mixing,
(iv) preparing a solution comprising insulin aspart having pH of about 6 to about 7, and
(v) mixing the suspension of step (iii) and solution of step (iv) to obtain the stable biphasic pharmaceutical composition.

In another embodiment of the invention, there is provided a method of preparing a stable biphasic pharmaceutical composition comprising: (a) insulin glargine in a suspension form, (b) insulin human, (c) at least two amino acids in a weight ratio of 1:2, and optionally (d) one or more pharmaceutically acceptable excipients, wherein the pharmaceutical composition has a pH between about 6.0 to about 8.0, wherein the said method comprises steps of:
(i) preparing a solution comprising insulin glargine having pH adjusted in between about 3 and about 4,
(ii) preparing a solution of sodium hydroxide (1N) and adding it to the solution of step (i) to obtain a suspension of insulin glargine having pH of about 7 to about 8,
(iii) preparing a solution of at least two amino acids and adding it into the suspension of step (ii) with mixing,
(iv) preparing a solution comprising insulin human having pH of about 6 to about 7, and
(v) mixing the suspension of step (iii) and solution of step (iv) to obtain the stable biphasic pharmaceutical composition.

In another embodiment of the invention, there is provided a method of treating type I and type II diabetes mellitus in a patient in need thereof, said method comprises administering to said patient a pharmaceutical composition comprising: (a) insulin glargine, (b) at least two amino acids in a weight ratio of about 1:2, and optionally (c) one or more pharmaceutically acceptable excipients.

In another embodiment of the invention, there is provided a method of treating type I and type II diabetes mellitus in a patient in need thereof, said method comprises administering to said patient a pharmaceutical composition comprising: (a) insulin glargine, (b) at least two amino acids in a weight ratio of about 1:2, and optionally (c) one or more pharmaceutically acceptable excipients, wherein the pharmaceutical composition is compatible when mixed with another insulin, insulin analogue or derivative having isoelectric point between 4.0 and 5.7.

In another embodiment of the invention, there is provided a method of treating type I and type II diabetes mellitus in a patient in need thereof, said method comprises administering to said the patient a stable biphasic pharmaceutical composition comprising: (a) insulin glargine in a suspension form, (b) another insulin, insulin analogue or derivative having isoelectric point between 4.0 and 5.7, (c) at least two amino acids in a weight ratio of about 1:2, and optionally (d) one or more pharmaceutically acceptable excipients, wherein the pharmaceutical composition has a pH between about 6.0 to about 8.0.

In another embodiment of the invention, there is provided a kit comprising first and second component, said first component comprises a pharmaceutical composition comprising: (a) insulin glargine, (b) at least two amino acids in a weight ratio of about 1:2, and optionally (c) one or more pharmaceutically acceptable excipients, and said second component comprises a pharmaceutical composition comprising: (a) another insulin, insulin analogue or derivative having isoelectric point between 4.0 and 5.7 and (b) one or more pharmaceutically acceptable excipients, and wherein the pharmaceutical composition of the first component is compatible when mixed with the pharmaceutical composition of second component.

In another embodiment of the invention, there is provided a kit comprising first and second component, said first component comprises a pharmaceutical composition comprising: (a) insulin glargine, (b) arginine and isoleucine in a weight ratio of about 1:2, and optionally (c) one or more pharmaceutically acceptable excipients, and said second component comprises a pharmaceutical composition comprising: (a) another insulin, insulin analogue or derivative having isoelectric point between 4.0 and 5.7 and (b) one or more pharmaceutically acceptable excipients, and wherein the pharmaceutical composition of the first component is compatible when mixed with the pharmaceutical composition of second component, and wherein the pharmaceutical composition of the first component can be mixed with the pharmaceutical composition of second component before injecting into a patient in the proportion, as percentage, 85:15, or 80:20, or 75:25, or 70:30, 65:35, or 60:40, or 55:45.

In another embodiment of the invention, there is provided a kit comprising first and second component, said first component comprises a pharmaceutical composition comprising: (a) insulin glargine, (b) arginine and isoleucine in a weight ratio of about 1:2, and optionally (c) one or more pharmaceutically acceptable excipients, and said second component comprises a pharmaceutical composition comprising: (a) insulin aspart and (b) one or more pharmaceutically acceptable excipients, and wherein the pharmaceutical composition of the first component is compatible when mixed with the pharmaceutical composition of second component, and wherein the pharmaceutical composition of the first component can be mixed with the pharmaceutical composition of second component before injecting into a patient in the proportion, as percentage, 85:15, or 80:20, or 75:25, or 70:30, 65:35, or 60:40, or 55:45.

In another embodiment of the invention, there is provided a kit comprising first and second component, said first component comprises a pharmaceutical composition comprising: (a) insulin glargine, (b) arginine and isoleucine in a weight ratio of about 1:2, and optionally (c) one or more pharmaceutically acceptable excipients, and said second component comprises a pharmaceutical composition comprising: (a) insulin human and (b) one or more pharmaceutically acceptable excipients, and wherein the pharmaceutical composition of the first component is compatible when mixed with the pharmaceutical composition of second component, and wherein the pharmaceutical composition of the first component can be mixed with the pharmaceutical composition of second component before injecting into a patient in the proportion, as percentage, 85:15, or 80:20, or 75:25, or 70:30, 65:35, or 60:40, or 55:45.

While the invention has been described in term of its specific embodiments, certain modification and equivalents will be apparent to those skilled in the art and are intended to be included within the scope of the invention.

### EXAMPLES

### Example 1: Preparation of compatible insulin glargine (100 IU/ml and 200 IU/ml) solution composition

Example 1 of invention elaborates composition and manufacturing process for composition of compatible insulin glargine having a pH of 3.5±0.2 that is ready to mix (before injection) with insulin aspart solution or insulin human solution having a pH of 7.2±0.2, in the proportion ranging from, as percentage, 90:10 to 50:50 (insulin glargine:insulin aspart or insulin human).

Manufacturing process:

(a) Compatible insulin glargine solution 100 IU/ml and 200 IU/ml, 500 ml:

Insulin glargine solution. 500 ml
Each ml contains:

| | | | | |
|---|---|---|---|---|
| Insulin Glargine | 3.6 | mg (100 IU) | 7.2 | mg (200 IU) |
| 85% Glycerol | 10 | mg | 10 | mg |
| Zinc chloride | 30 | μg | 60 | μg |
| m-Cresol | 2.7 | mg | 2.7 | mg |
| Glycine | 100 | mM | 100 | mM |
| L-Arginine | 7.14 | mM | 7.14 | mM |
| L-Isoleucine | 14.28 | mM | 14.28 | mM |

Defined quantity of m-cresol and glycerol (85%) were dissolved in 10% final batch size of water for injection to prepare a preservative solution. The insulin glargine and zinc chloride were suspended in 5% final batch size of water for injection to obtain slurry. To prepare a solution of insulin glargine, hydrochloric acid (1N) was added in the insulin glargine slurry at 1 ml/g of insulin glargine concentration (v/w). The defined quantity of glycine was then added in the dissolved insulin glargine solution and followed by addition of the preservative solution. To this, while stirring, calculated quantities of L-arginine and L-isoleucine (in the weight ratio of 1:2) were added. The solution volume was made up to 80% of final formulation batch size with water for injection. The pH of solution was further adjusted to 3.5±0.2 using hydrochloric acid (1N) or sodium hydroxide (1N). The final volume was made up to 100% with water for injection. The resultant insulin glargine solution was sterile filtered using 0.2 μ PES membrane filter and collected in a filling tank.

(b) Insulin aspart solution 100 IU/ml and 200 IU/ml, 500 ml:

Insulin aspart solution, 500 ml
Each ml contains:

| | | | | |
|---|---|---|---|---|
| Insulin Aspart | 3.6 | mg (100 IU) | 7.2 | mg (200 IU) |
| Phenol | 1.5 | mg | 1.5 | mg |
| 85% Glycerol | 20 | mg | 20 | mg |
| Zinc chloride | 30 | μg | 60 | μg |
| m-Cresol | 1.72 | mg | 1.72 | mg |
| di-Sodium hydrogen | 1.25 | mg | 1.25 | mg |
| phosphate dihydrate | | | | |

In a separate container, a defined quantity of phenol, m-cresol, glycerol (85%) and di-Sodium hydrogen phosphate dihydrate were dissolved in 10% of final batch size volume of water for injection to form a preservative solution. The insulin aspart and zinc chloride were suspended in 5% final batch size of water for injection to obtain slurry. To prepare a solution of insulin aspart, hydrochloric acid (1N) was added in the insulin aspart slurry at 1 ml/g of insulin aspart concentration (v/w). The prepared preservative solution was further added to above insulin aspart solution. The volume of solution from above step was made up with sterile water for injection to 80% of final batch size. The solution pH was adjusted to 7.2±0.2 by using sodium hydroxide (1N) solution to prepare the insulin aspart solution. Post pH adjustment, volume of the above solution is made up with water for injection to 100% of final batch. Prepared insulin aspart solution was filtered using 0.2 μ PES membrane filter and collected in another filling tank.

(c) Insulin human solution 100 IU/ml and 200 IU/ml, 500 ml:

Insulin human solution, 500 ml
Each ml contains:

| | | | | |
|---|---|---|---|---|
| Insulin human | 3.6 | mg (100 IU) | 7.2 | mg (200 IU) |
| Citric acid monohydrate | 0.0021 | mg | 0.0021 | mg |
| 85% Glycerol | 20 | mg | 20 | mg |
| Zinc chloride | 30 | μg | 60 | μg |
| m-Cresol | 2.5 | mg | 2.5 | mg |
| Tri-Sodium citrate | 0.19 | mg | 0.19 | mg |

In a separate container, defined quantity of m-cresol, glycerol (85%), citric acid monohydrate and tri- sodium citrate were dissolved in 10% of final batch size volume of water for injection to form a preservative solution. The insulin human and zinc chloride were suspended in 5% final batch size of water for injection to obtain slurry. To prepare a solution of insulin human, hydrochloric acid (1N) was added in the insulin human slurry at 1 ml/g of insulin human concentration (v/w). The prepared preservative solution was further added to above insulin human solution. The volume of solution from above step was made up with water for injection to 80% of final batch size. The solution pH was adjusted to 7.2±0.2 by using sodium hydroxide (1N) solution to prepare the insulin human solution. Post pH adjustment, volume of the above solution is made up with water for injection to 100% of final batch. Prepared insulin human solution was filtered using 0.2 μ PES membrane filter and collected in another filling tank.

(d) Preparation of a premix solution (before injection) containing insulin glargine and insulin aspart or insulin human (70/30):

The solution formulations of insulin glargine and insulin aspart or insulin human are ready to be mixed with each other in the proportion ranging from, as percentage, 90:10 to 50:50. Since these solutions are mixed before injection, therefore solutions are filled separately in the suitable container closure systems and can be mixed just before the injection in a suitable sterile container system. For instance, the final solution containing insulin glargine and insulin aspart or insulin human (100 IU/ml, 70/30) was prepared by mixing 70 ml of insulin glargine solution (100 IU/ml) and 30 ml of insulin aspart or insulin human solution (100 IU/ml) and the final solution containing insulin glargine and insulin aspart or insulin human (200 IU/ml, 70/30) was prepared by mixing 70 ml of insulin glargine solution (200 IU/ml) and 30 ml of insulin aspart or insulin human solution (200 IU/ml) in a suitable container has following composition:

| Each ml contains: | 100 IU/ml | | 200 IU/ml | | 100 IU/ml | | 200 IU/ml | |
|---|---|---|---|---|---|---|---|---|
| Insulin Glargine | 70 | IU | 140 | IU | 70 | IU | 140 | IU |
| Insulin Aspart | 30 | IU | 60 | IU | — | | — | |
| Insulin Human | — | | — | | 30 | IU | 60 | IU |
| Phenol | 0.45 | mg | 0.45 | mg | — | | — | |
| 85% Glycerol | 13 | mg | 13 | mg | 13 | mg | 13 | mg |
| Zinc chloride | 30 | μg | 60 | μg | 30 | μg | 60 | μg |
| m-Cresol | 2.4 | mg | 2.4 | mg | 2.64 | mg | 2.64 | mg |
| di-Sodium hydrogen | 0.375 | mg | 0.375 | mg | — | | — | |
| phosphate dihydrate | | | | | | | | |
| Tri-sodium citrate | — | | — | | 0.057 | mg | 0.057 | mg |
| Citric acid monohydrate | — | | — | | 0.0021 | mg | 0.0021 | mg |
| Glycine | 70 | mM | 70 | mM | 70 | mM | 70 | mM |
| L-Arginine | 5 | mM | 5 | mM | 5 | mM | 5 | mM |
| L-Isoleucine | 10 | mM | 10 | mM | 10 | mM | 10 | mM |

| | | | | |
|---|---|---|---|---|
| pH | 3.5 ± 0.2 | 3.5 ± 0.2 | 3.5 ± 0.2 | 3.5 ± 0.2 |

### EXAMPLE 2: Comparative solution compositions of insulin glargine and insulin aspart (200 IU/ml) and stability studies

Three comparative compositions were made: composition devoid of L-isoleucine (2A), and composition devoid of L-arginine (2B) and altering the weight ratio of L-arginine and L-isoleucine that is 1:1 (2C). These compositions were compared with invention composition of Example 1 (solution composition of insulin glargine and insulin aspart, 200 IU/ml).

Comparative compositions

| | | | | |
|---|---|---|---|---|
| | | Comparative | Comparative | Comparative |
| | | composition | composition | composition |
| Ingredients | Example 1 | A | B | C |

Each ml contains:

| | | | | | | | | |
|---|---|---|---|---|---|---|---|---|
| Insulin | 140 | IU | 140 | IU | 140 | IU | 140 | IU |
| Glargine | | | | | | | | |
| Insulin Aspart | 60 | IU | 60 | IU | 60 | IU | 60 | IU |
| Phenol | 0.45 | mg | 0.45 | mg | 0.45 | mg | 0.45 | mg |
| 85% Glycerol | 13 | mg | 13 | mg | 13 | mg | 13 | mg |
| Zinc chloride | 60 | μg | 60 | μg | 60 | μg | 60 | μg |
| m-Cresol | 2.4 | mg | 2.4 | mg | 2.4 | mg | 2.4 | mg |
| di-Sodium | 0.375 | mg | 0.375 | mg | 0.375 | mg | 0.375 | mg |
| hydrogen | | | | | | | | |
| phosphate | | | | | | | | |
| dihydrate | | | | | | | | |
| Glycine | 70 | mM | 70 | mM | 70 | mM | 70 | mM |

| | | | | | | | |
|---|---|---|---|---|---|---|---|
| L-Arginine | 5 | mM | 5 | mM | — | 5 | mM |

| | | | | | | | |
|---|---|---|---|---|---|---|---|
| L-Isoleucine | 10 | mM | — | 10 | mM | 5 | mM |

| | | | | |
|---|---|---|---|---|
| pH | 3.5 ± 0.2 | 3.5 ± 0.2 | 3.5 ± 0.2 | 3.5 ± 0.2 |

In Example 1 compositions and comparative compositions, where the contact time (before injection in a syringe) for two components (insulin glargine and insulin aspart) is expected to be less than 10 seconds to about 5 minutes, the stability of acid labile insulin aspart part was carried up to 24 hours at 25° C. and relative humidity of 60%. Before analysis, to separate the two insulin's components from each other, samples were passed through cation-exchange column. This was followed by analysis of related impurities using—“Method for insulin aspart related proteins and method for high molecular weight proteins—Indian Pharmacopoeia 2014”

Stability of insulin aspart with insulin glargine in solution
of example 1 and comparative compositions (pH 3.5 ±
0.2, up to 24 h, at 25° C. and relative humidity of 60%)

| | | High | | | | |
|---|---|---|---|---|---|---|
| | | Molecular | Related impurities | | | |
| | Time in | weight | | A21 Asp | | |
| Compositions | Hours | impurities | B28 isoAsp | and B3 Asp | B3 isoAsp | Others |
| Example 1 | Initial | 0.01 | 0.01 | 0.08 | 0.02 | 0.09 |
| | 2 | 0.01 | 0.01 | 0.11 | 0.03 | 0.14 |
| | 4 | 0.03 | 0.03 | 0.23 | 0.05 | 0.29 |
| | 8 | 0.04 | 0.08 | 0.41 | 0.1 | 0.53 |
| | 24 | 0.12 | 0.14 | 0.96 | 0.22 | 1.18 |
| Comparative | Initial | 0.01 | 0.02 | 0.11 | 0.03 | 0.16 |
| Example 2A | 2 | 0.06 | 0.04 | 0.38 | 0.05 | 0.34 |
| | 4 | 0.13 | 0.06 | 0.57 | 0.09 | 0.74 |
| | 8 | 0.32 | 0.09 | 0.86 | 0.19 | 1.34 |
| | 24 | 0.93 | 0.18 | 1.25 | 0.34 | 2.5 |
| Comparative | Initial | 0.01 | 0.04 | 0.09 | 0.07 | 0.23 |
| Example 2B | 2 | 0.04 | 0.07 | 0.14 | 0.11 | 0.4 |
| | 4 | 0.1 | 0.1 | 0.34 | 0.16 | 0.85 |
| | 8 | 0.24 | 0.16 | 0.77 | 0.31 | 1.51 |
| | 24 | 0.78 | 0.24 | 1.64 | 0.45 | 2.77 |
| Comparative | Initial | 0.01 | 0.03 | 0.09 | 0.05 | 0.1 |
| Example 2C | 2 | 0.03 | 0.05 | 0.17 | 0.09 | 0.19 |
| | 4 | 0.09 | 0.08 | 0.35 | 0.13 | 0.36 |
| | 8 | 0.2 | 0.13 | 0.62 | 0.19 | 0.62 |
| | 24 | 0.56 | 0.18 | 1.18 | 0.27 | 1.95 |

As evident from the stability data that, the formation of high molecular weight impurities and related impurities with respect to insulin aspart in a premix with insulin glargine are more in comparative composition 2A, 2B and 2C than in Example 1 composition of the invention.

### EXAMPLE 3: Comparative solution compositions of insulin glargine and insulin human (200 IU/ml) and stability studies

Three comparative compositions were made: composition devoid of L-isoleucine (3A), and composition devoid of L-arginine (3B) and altering the weight ratio of L-arginine and L-isoleucine that is 1:1 (3C). These compositions were compared with invention composition of example 1 (solution composition of insulin glargine and insulin human, 200 IU/ml).

Comparative compositions

| | | | | |
|---|---|---|---|---|
| | | Comparative | Comparative | Comparative |
| | | composition | composition | composition |
| Ingredients | Example 1 | A | B | C |

Each ml contains:

| | | | | | | | | |
|---|---|---|---|---|---|---|---|---|
| Insulin Glargine | 140 | IU | 140 | IU | 140 | IU | 140 | IU |
| Insulin Human | 60 | IU | 60 | IU | 60 | IU | 60 | IU |
| Glycine | 70 | mM | 70 | mM | 70 | mM | 70 | mM |
| 85% Glycerol | 13 | mg | 13 | mg | 13 | mg | 13 | mg |
| Zinc chloride | 60 | μg | 60 | μg | 60 | μg | 60 | μg |
| m-Cresol | 2.64 | mg | 2.64 | mg | 2.64 | mg | 2.64 | mg |
| Tri-sodium citrate | 0.057 | mg | 0.057 | mg | 0.057 | mg | 0.057 | mg |
| Citric acid monohydrate | 0.0021 | mg | 0.0021 | mg | 0.0021 | mg | 0.0021 | mg |

| | | | | | | | |
|---|---|---|---|---|---|---|---|
| L-Arginine | 5 | mM | 5 | mM | — | 5 | mM |

| | | | | | | | |
|---|---|---|---|---|---|---|---|
| L-Isoleucine | 10 | mM | — | 10 | mM | 5 | mM |

| | | | | |
|---|---|---|---|---|
| pH | 3.5 ± 0.2 | 3.5 ± 0.2 | 3.5 ± 0.2 | 3.5 ± 0.2 |

In example 1 compositions and comparative compositions, where the contact time (before injection in a syringe) for two components (insulin glargine and insulin human) is expected to be less than 10 seconds to about 5 minutes, the stability of acid labile insulin human part was carried up to 24 hours at 25° C. and relative humidity of 60%. Before analysis, to separate the two insulin's components from each other, samples were passed through cation-exchange column. This was followed by analysis of related impurities using—“Method for insulin human related proteins and method for high molecular weight proteins—European Pharmacopoeia 8^{th} edition, Pg 2491-2493”.

Stability of insulin human with insulin glargine in solution
of example 1 and comparative compositions (pH 3.5 ±
0.2, up to 24 h, at 25° C. and relative humidity of 60%)

| | |
|---|---|
| High | Related Impurities |

| | | Molecular | A-21 | |
|---|---|---|---|---|
| | Time in | weight | desamido | |
| Compositions | Hours | impurities | insulin | Others |
| Example 1 | Initial | 0.01 | 0.3 | 0.07 |
| | 2 | 0.01 | 0.11 | 0.13 |
| | 4 | 0.03 | 0.23 | 0.30 |
| | 8 | 0.04 | 0.14 | 0.68 |
| | 24 | 0.08 | 0.41 | 1.8 |
| Comparative | Initial | 0.01 | 0.06 | 0.18 |
| Example 3A | 2 | 0.04 | 0.18 | 0.37 |
| | 4 | 0.11 | 0.42 | 0.79 |
| | 8 | 0.25 | 0.64 | 1.42 |
| | 24 | 0.71 | 0.87 | 2.74 |
| Comparative | Initial | 0.01 | 0.07 | 0.21 |
| Example 3B | 2 | 0.05 | 0.12 | 0.42 |
| | 4 | 0.11 | 0.30 | 0.82 |
| | 8 | 0.29 | 0.62 | 1.59 |
| | 24 | 0.86 | 1.14 | 2.92 |
| Comparative | Initial | 0.01 | 0.07 | 0.13 |
| Example 3C | 2 | 0.05 | 0.14 | 0.44 |
| | 4 | 0.12 | 0.34 | 0.91 |
| | 8 | 0.29 | 0.57 | 1.72 |
| | 24 | 0.68 | 0.92 | 3.14 |

As evident from the stability data that, the formation of high molecular weight impurities and related impurities with respect to insulin human in a premix with insulin glargine are more in comparative composition 3A, 3B and 3C than in Example 1 composition of the invention.

### EXAMPLE 4: Biphasic composition of insulin glargine and insulin aspart or insulin human (100 IU/m1 and 200 IU/ml suspension)

Example 4 of invention elaborates composition and manufacturing process for composition of compatible insulin glargine suspension having a pH of 7.2±0.2 formulated with insulin aspart or insulin human solution having a pH 7.2±0.2. The insulin glargine and insulin aspart or insulin human can be mixed in the proportion ranging from, as percentage, 90:10 to 50:50 (insulin glargine:insulin aspart or insulin human).

Manufacturing process:

(a) Compatible insulin glargine suspension, 700 ml:

Insulin glargine suspension, 700 ml
Each ml contains:

| | | | | |
|---|---|---|---|---|
| Insulin Glargine | 3.6 | mg (100 IU) | 7.2 | mg (200 IU) |
| 85% Glycerol | 10 | mg | 10 | mg |
| Zinc chloride | 30 | μg | 60 | μg |
| m-Cresol | 2.7 | mg | 2.7 | mg |
| Glycine | 100 | mM | 100 | mM |
| L-Arginine | 7.14 | mM | 7.14 | mM |
| L-Isoleucine | 14.28 | mM | 14.28 | mM |

(i) Defined quantity of m-cresol and glycerol (85%) were dissolved in 10% final batch size of water for injection to prepare a preservative solution. The insulin glargine and zinc chloride were suspended in 5% final batch size of water for injection to obtain slurry. To prepare a solution of insulin glargine, hydrochloric acid (1N) was added in the insulin glargine slurry at 1 ml/g of insulin glargine concentration (v/w). The defined quantity of glycine was then added in the dissolved insulin glargine solution and followed by addition of the preservative solution. The solution volume was made up to 30% of final formulation batch size with water for injection. The pH of solution was at this stage was 3.5±0.2. The resultant insulin glargine solution was sterile filtered using 0.2 μ PES membrane filter and collected in sterile container X.

(ii) Defined quantity of sodium hydroxide (1N) solution was filtered using 0.2 μ PES membrane filter and added slowly in a sterile container X, where insulin glargine solution was previously collected. This activity was performed to adjust the pH of insulin glargine solution to 7.2±0.2. This change in pH resulted in formation of insulin glargine suspension of controlled particle size. The volume of the suspension was made up to 60% final batch size.

(iii) Arginine:Isoleucine solution—In a separate container 8% final batch size volume of water for injection was taken. While stirring, calculated quantities of L-Arginine and L-Isoleucine (1:2 weight ratio) were added in the water for injection. The solution was mixed at 300 rpm till the complete dissolution of aforementioned amino acids. The solution pH was adjusted to 7.2±0.2 using hydrochloric acid (1N) or sodium hydroxide (1N). The prepared solution was filtered using 0.2 μ PES membrane and added in a sterile container X with mixing. The volume of the parts present in sterile container X was made up to 70% final batch size.

(b) Insulin aspart solution, 300 ml:

Insulin aspart solution, 300 ml
Each ml contains:

| | | | | |
|---|---|---|---|---|
| Insulin Aspart | 3.6 | mg (100 IU) | 7.2 | mg (200 IU) |
| Phenol | 1.5 | mg | 1.5 | mg |
| 85% Glycerol | 20 | mg | 20 | mg |
| Zinc chloride | 30 | μg | 60 | μg |
| m-Cresol | 1.72 | mg | 1.72 | mg |
| di-Sodium hydrogen | 1.25 | mg | 1.25 | mg |
| phosphate dihydrate | | | | |

In a separate container, a defined quantity of phenol, m-cresol, glycerol (85%) and di-Sodium hydrogen phosphate dihydrate were dissolved in 10% of final batch size volume of water for injection to form a preservative solution. The insulin aspart and zinc chloride were suspended in 5% final batch size of water for injection to obtain slurry. To prepare a solution of insulin aspart, hydrochloric acid (1N) was added in the insulin aspart slurry at 1 ml/g of insulin aspart concentration (v/w). The prepared preservative solution was further added to above insulin aspart solution. The volume of solution from above step was made up with sterile water for injection to 25% of final batch size. The solution pH was adjusted to 7.2±0.2 by using sodium hydroxide (1N) solution to prepare the insulin aspart solution. Post pH adjustment, volume of the above solution is made up with water for injection to 30% of final batch.

(c) Insulin human solution, 300 ml:

Insulin human solution, 300 ml
Each ml contains:

| | | | | |
|---|---|---|---|---|
| Insulin human | 3.6 | mg (100 IU) | 7.2 | mg (200 IU) |
| Citric acid monohydrate | 0.0021 | mg | 0.0021 | mg |
| 85% Glycerol | 20 | mg | 20 | mg |
| Zinc chloride | 30 | μg | 60 | μg |
| m-Cresol | 2.5 | mg | 2.5 | mg |
| Tri-Sodium citrate | 0.19 | mg | 0.19 | mg |

In a separate container, defined quantity of m-cresol, glycerol (85%), citric acid monohydrate and tri- sodium citrate were dissolved in 10% of final batch size volume of water for injection to form a preservative solution. The insulin human and zinc chloride were suspended in 5% final batch size of water for injection to obtain slurry. To prepare a solution of insulin human, hydrochloric acid (1N) was added in the insulin human slurry at 1 ml/g of insulin human concentration (v/w). The prepared preservative solution was further added to above insulin human solution. The volume of solution from above step was made up with water for injection to 25% of final batch size. The solution pH was adjusted to 7.2±0.2 by using sodium hydroxide (1N) solution to prepare the insulin human solution. Post pH adjustment, volume of the above solution is made up with water for injection to 30% of final batch.

(d) Preparation of a biphasic composition containing insulin glargine and insulin aspart or insulin human (70/30):

The suspension of insulin glargine and solution of insulin aspart or insulin human can be mixed with each other in the proportion ranging from, as percentage, 90:10 to 50:50. The final suspension containing insulin glargine and insulin aspart or insulin human (100 IU/ml, 70/30) was prepared by mixing 700 ml of insulin glargine suspension (100 IU/ml) and 300 ml of insulin aspart or insulin human solution (100 IU/ml) and the final suspension containing insulin glargine and insulin aspart or insulin human (200 IU/ml, 70/30) was prepared by mixing 700 ml of insulin glargine suspension (200 IU/ml) and 300 ml of insulin aspart or insulin human solution (200 IU/ml) in a suitable container and has following composition:

| Each ml contains: | 100 IU/ml | | 200 IU/ml | | 100 IU/ml | | 200 IU/ml | |
|---|---|---|---|---|---|---|---|---|
| Insulin Glargine | 70 | IU | 140 | IU | 70 | IU | 140 | IU |
| Insulin Aspart | 30 | IU | 60 | IU | — | | — | |
| Insulin Human | — | | — | | 30 | IU | 60 | IU |
| Phenol | 0.45 | mg | 0.45 | mg | — | | — | |
| 85% Glycerol | 13 | mg | 13 | mg | 13 | mg | 13 | mg |
| Zinc chloride | 30 | μg | 60 | μg | 30 | μg | 60 | μg |
| m-Cresol | 2.4 | mg | 2.4 | mg | 2.64 | mg | 2.64 | mg |
| di-Sodium hydrogen | 0.375 | mg | 0.375 | mg | — | | — | |
| phosphate dihydrate | | | | | | | | |
| Tri-sodium citrate | — | | — | | 0.057 | mg | 0.057 | mg |
| Citric acid monohydrate | — | | — | | 0.0021 | mg | 0.0021 | mg |
| Glycine | 70 | mM | 70 | mM | 70 | mM | 70 | mM |
| L-Arginine | 5 | mM | 5 | mM | 5 | mM | 5 | mM |
| L-Isoleucine | 10 | mM | 10 | mM | 10 | mM | 10 | mM |

| | | | | |
|---|---|---|---|---|
| pH | 7.2 ± 0.2 | 7.2 ± 0.2 | 7.2 ± 0.2 | 7.2 ± 0.2 |

### EXAMPLE 5: Comparative biphasic compositions containing insulin glargine and insulin aspart (200 IU/ml) and stability studies

Three comparative biphasic compositions were made: composition devoid of L-isoleucine (5A), and composition devoid of L-arginine (5B) and altering the weight ratio of L-arginine and L-isoleucine that is 1:1 (5C). These compositions were compared with invention composition of example 4 (Biphasic composition of insulin glargine and insulin aspart, 200 IU/ml).

Comparative compositions

| | | | | |
|---|---|---|---|---|
| | | Comparative | Comparative | Comparative |
| | | composition | composition | composition |
| Ingredients | Example 4 | A | B | C |

Each ml contains:

| | | | | | | | | |
|---|---|---|---|---|---|---|---|---|
| Insulin | 140 | IU | 140 | IU | 140 | IU | 140 | IU |
| Glargine | | | | | | | | |
| Insulin Aspart | 60 | IU | 60 | IU | 60 | IU | 60 | IU |
| Phenol | 0.45 | mg | 0.45 | mg | 0.45 | mg | 0.45 | mg |
| 85% Glycerol | 13 | mg | 13 | mg | 13 | mg | 13 | mg |
| Zinc chloride | 60 | μg | 60 | μg | 60 | μg | 60 | μg |
| m-Cresol | 2.4 | mg | 2.4 | mg | 2.4 | mg | 2.4 | mg |
| di-Sodium | 0.375 | mg | 0.375 | mg | 0.375 | mg | 0.375 | mg |
| hydrogen | | | | | | | | |
| phosphate | | | | | | | | |
| dihydrate | | | | | | | | |
| Glycine | 70 | mM | 70 | mM | 70 | mM | 70 | mM |

| | | | | | | | |
|---|---|---|---|---|---|---|---|
| L-Arginine | 5 | mM | 5 | mM | — | 5 | mM |

| | | | | | | | |
|---|---|---|---|---|---|---|---|
| L-Isoleucine | 10 | mM | — | 10 | mM | 5 | mM |

| | | | | |
|---|---|---|---|---|
| pH | 7.2 ± 0.2 | 7.2 ± 0.2 | 7.2 ± 0.2 | 7.2 ± 0.2 |

In Example 4 compositions and comparative compositions, where the contact time for two components (insulin glargine and insulin aspart) is expected to be at least 6 months, the stability of insulin glargine part was carried up to 6 months at 25° C. and relative humidity of 60% (accelerated stability condition; considered as indicator of product stability at 2-8° C. up to 24 months). Before analysis, to separate the two insulin's components from each other, samples were passed through cation-exchange column. This was followed by analysis of related impurities using—“Method for insulin glargine related proteins and method for high molecular weight proteins—United States Pharmacopoeia 38^{th} edition, Pg 3876-3878”.

Stability of insulin glargine in biphasic composition of example
4 with insulin aspart and comparative compositions (pH 7.2 ±
0.2, up to 6 months, at 25° C. and relative humidity of 60%)

| | | High | |
|---|---|---|---|
| | | Molecular | |
| | Time in | weight | Related |
| Compositions | Month | impurities | Impurities |
| Example 4 | Initial | 0.01 | 0.03 |
| | 1 | 0.05 | 0.05 |
| | 2 | 0.09 | 0.07 |
| | 4 | 0.13 | 0.11 |
| | 6 | 0.18 | 0.19 |
| Comparative | Initial | 0.01 | 0.05 |
| Example 5A | 1 | 0.12 | 0.15 |
| | 2 | 0.23 | 0.42 |
| | 4 | 0.35 | 0.89 |
| | 6 | 0.42 | 1.64 |
| Comparative | Initial | 0.01 | 0.08 |
| Example 5B | 1 | 0.14 | 0.21 |
| | 2 | 0.26 | 0.43 |
| | 4 | 0.4 | 0.95 |
| | 6 | 0.57 | 1.78 |
| Comparative | Initial | 0.01 | 0.05 |
| Example 5C | 1 | 0.1 | 0.11 |
| | 2 | 0.23 | 0.21 |
| | 4 | 0.4 | 0.46 |
| | 6 | 0.49 | 0.73 |

As evident from the stability data that, the formation of high molecular weight impurities and related impurities with respect to insulin glargine in a biphasic composition are more in comparative composition 5A, 5B and 5C than in Example 4 composition of the invention.

### EXAMPLE 6: Comparative biphasic compositions of insulin glargine and insulin human (2001U/ml) and stability studies

Three comparative biphasic compositions were made: composition devoid of L-isoleucine (6A), and composition devoid of L-arginine (6B) and altering the weight ratio of L-arginine and L-isoleucine that is 1:1 (6C). These compositions were compared with invention composition of example 4 (biphasic composition of insulin glargine and insulin human, 200 IU/ml).

Comparative compositions

| | | | | |
|---|---|---|---|---|
| | | Comparative | Comparative | Comparative |
| | | composition | composition | composition |
| Ingredients | Example 4 | A | B | C |

Each ml contains:

| | | | | | | | | |
|---|---|---|---|---|---|---|---|---|
| Insulin Glargine | 140 | IU | 140 | IU | 140 | IU | 140 | IU |
| Insulin Human | 60 | IU | 60 | IU | 60 | IU | 60 | IU |
| Glycine | 70 | mM | 70 | mM | 70 | mM | 70 | mM |
| 85% Glycerol | 13 | mg | 13 | mg | 13 | mg | 13 | mg |
| Zinc chloride | 60 | μg | 60 | μg | 60 | μg | 60 | μg |
| m-Cresol | 2.64 | mg | 2.64 | mg | 2.64 | mg | 2.64 | mg |
| Tri-sodium citrate | 0.057 | mg | 0.057 | mg | 0.057 | mg | 0.057 | mg |
| Citric acid monohydrate | 0.0021 | mg | 0.0021 | mg | 0.0021 | mg | 0.0021 | mg |

| | | | | | | | |
|---|---|---|---|---|---|---|---|
| L-Arginine | 5 | mM | 5 | mM | — | 5 | mM |

| | | | | | | | |
|---|---|---|---|---|---|---|---|
| L-Isoleucine | 10 | mM | — | 10 | mM | 5 | mM |

| | | | | |
|---|---|---|---|---|
| pH | 7.2 ± 0.2 | 7.2 ± 0.2 | 7.2 ± 0.2 | 7.2 ± 0.2 |

In Example 4 compositions and comparative compositions, where the contact time for two components (insulin glargine and insulin human) is expected to be at least 6 months, the stability of insulin glargine part was carried up to 6 months at 25° C. and relative humidity of 60% (accelerated stability condition; considered as indicator of product stability at 2-8° C. up to 24 months). Before analysis, to separate the two insulin's components from each other, samples were passed through cation-exchange column. This was followed by analysis of related impurities using—“Method for insulin glargine related proteins and method for high molecular weight proteins—United States Pharmacopoeia 38^{th} edition, Pg 3876-3878”.

Stability of insulin glargine in biphasic composition of Example
4 with insulin human and comparative compositions (pH 7.2 ±
0.2, up to 6 months, at 25° C. and relative humidity of 60%)

| | | High | |
|---|---|---|---|
| | | Molecular | |
| | Time in | weight | Related |
| Compositions | Month | impurities | Impurities |
| Example 4 | Initial | 0.01 | 0.04 |
| | 1 | 0.04 | 0.06 |
| | 2 | 0.08 | 0.8 |
| | 4 | 0.11 | 0.13 |
| | 6 | 0.17 | 0.21 |
| Comparative | Initial | 0.03 | 0.04 |
| Example 6A | 1 | 0.14 | 0.13 |
| | 2 | 0.25 | 0.39 |
| | 4 | 0.34 | 0.85 |
| | 6 | 0.44 | 1.59 |
| Comparative | Initial | 0.02 | 0.07 |
| Example 6B | 1 | 0.11 | 0.23 |
| | 2 | 0.23 | 0.47 |
| | 4 | 0.42 | 0.93 |
| | 6 | 0.55 | 1.72 |
| Comparative | Initial | 0.01 | 0.04 |
| Example 6C | 1 | 0.12 | 0.10 |
| | 2 | 0.25 | 0.22 |
| | 4 | 0.43 | 0.45 |
| | 6 | 0.53 | 0.78 |

As evident from the stability data that, the formation of high molecular weight impurities and related impurities with respect to insulin glargine in a biphasic composition with insulin human are more in comparative composition 6A, 6B and 6C than in Example 4 composition of the invention.

1. A pharmaceutical composition comprising: (a) insulin glargine, (b) arginine and isoleucine in a weight ratio of 1:2, and optionally (c) one or more pharmaceutically acceptable excipients.

2. A pharmaceutical composition comprising: (a) insulin glargine, (b) arginine and isoleucine in a weight ratio of about 1:2, and optionally (c) one or more pharmaceutically acceptable excipients, wherein the pharmaceutical composition is compatible when mixed with another insulin having isoelectric point between 4.0 and 5.7.

3. A stable biphasic pharmaceutical composition comprising: (a) insulin glargine in a suspension form, (b) another insulin having isoelectric point between 4.0 and 5.7, (c) arginine and isoleucine in a weight ratio of 1:2, and optionally, (d) one or more pharmaceutically acceptable excipients, wherein the pharmaceutical composition has a pH between 6.0 to 8.0.

4. The pharmaceutical composition of claim 1 or 2, wherein the composition has pH in between 2 to 8.

5. The pharmaceutical composition of claim 2 or 3, wherein the another having isoelectric point between 4.0 and 5.7 is selected from the group comprising insulin human, insulin isophane (NPH), insulin lispro, insulin lispro protamine, insulin glulisine, insulin aspart, insulin aspart protamine, Gly(A21) human insulin, Gly(A21) Lys(B28) human insulin, Gly(A21) Lys(B28) Pro(B29) human insulin, Gly(A21) Asp(B28), insulin degludec and insulin detemir.

6.-7. (canceled)

8. The pharmaceutical composition of claim 1 or 2, wherein the pharmaceutical composition is in the form of solution.

9. The pharmaceutical composition of any one of claims 1-3, wherein insulin glargine is present in 40 IU to 500 IU.

10. The pharmaceutical composition of any one of claims 1-3, wherein the one or more pharmaceutically acceptable excipients comprise buffer, solubilising agent, isotonic agent, preservative, antioxidant, pH modifying agent or combination thereof

11. The pharmaceutical composition of claim 1 or 2, wherein the composition is stable for at least 24 hours at 25° C. and relative humidity of 60% after mixing with another insulin, insulin analogue or derivative having isoelectric point between 4.0 and 5.7.

12. The pharmaceutical composition of claim 3, wherein the composition is stable for at least 6 months at 25° C. and relative humidity of 60%.

13. A pharmaceutical composition comprising: (a) insulin glargine, (b) arginine and isoleucine in a weight ratio of about 1:2, and optionally (c) one or more pharmaceutically acceptable excipients, wherein the pharmaceutical composition is compatible when mixed with another insulin having isoelectric point between 4.0 and 5.7.

14. A stable biphasic pharmaceutical composition comprising: (a) insulin glargine in a suspension form, (b) another insulin having isoelectric point between 4.0 and 5.7, (c) arginine and isoleucine in a weight ratio of 1:2, and optionally (d) one or more pharmaceutically acceptable excipients, wherein the pharmaceutical composition has a pH between 6.0 to 8.0.

15. A method of preparing a pharmaceutical composition comprising: (a) insulin glargine, (b) another insulin having isoelectric point between 4.0 and 5.7, (c) arginine and isoleucine in a weight ratio of 1:2, and optionally (d) one or more pharmaceutically acceptable excipients, wherein the pharmaceutical composition has pH in between 3 to 4, and wherein the said method comprises steps of:
(i) preparing a solution comprising insulin glargine in acidic water for injection,
(ii) preparing a solution of arginine and isoleucine,
(iii) adding the solution of arginine and isoleucine in the solution of step (i) and adjusting the pH to 3 to 4 using a pH modifying agent,
(iv) preparing a solution comprising another insulin having isoelectric point between 4.0 and 5.7 having pH of 6 to 7, and
(v) mixing the solutions of step (iii) and (iv) to obtain the pharmaceutical composition.

16. A method of preparing a stable biphasic pharmaceutical composition comprising: (a) insulin glargine in a suspension form, (b) another insulin having isoelectric point between 4.0 and 5.7, (c) arginine and isoleucine in a weight ratio of 1:2, and optionally (d) one or more pharmaceutically acceptable excipients, wherein the pharmaceutical composition has a pH between 6.0 to 8.0, wherein the said method comprises steps of:
(i) preparing a solution comprising insulin glargine having pH adjusted in between 3 and 4,
(ii) preparing a solution of sodium hydroxide (1N) and adding it to the solution of step (i) to obtain a suspension of insulin glargine having pH of 7 to 8,
(iii) preparing a solution of arginine and isoleucine and adding it into the suspension of step (ii) with mixing,
(iv) preparing a solution comprising another insulin having isoelectric point between 4.0 and 5.7 having pH of 6 to 7, and
(v) mixing the suspension of step (iii) and solution of step (iv) to obtain the stable biphasic pharmaceutical composition.
